(19) United States
(12) Patent Application Publication
(10) Pub. No.: US 2022/0015999 A1
(43) Pub. Date: Jan. 20, 2022

(54) SUNSCREEN THAT PROMOTES OCEAN HEALTH

(71) Applicant: Coral Sunscreen LLC, Boulder, CO (US)

(72) Inventor: Emma Kraft, Boulder, CO (US)

(21) Appl. No.: 17/376,738

(22) Filed: Jul. 15, 2021

Related U.S. Application Data
(60) Provisional application No. 63/052,304, filed on Jul. 15, 2020.

(51) Int. Cl. A61K 8/29; A61K 8/27; A61K 8/19; A61Q 17/04

(52) U.S. Cl. CPC A61K 8/29; A61Q 17/04; A61K 8/19; A61K 8/27

(57) ABSTRACT

A sunscreen is disclosed that promotes ocean health, especially for coral reefs. The sunscreen includes an active sunscreen component, a delivery component, and one or more alkalinity enhancing components. The sunscreen component filters or blocks some amount of harmful solar radiation from affecting the wearer's skin and is preferably reef-safe and free from ingredients that pose health risks to wearers. The alkalinity enhancing components counteracts ocean acidification, or raises the pH, of the local environment, e.g., in the vicinity of reefs. The delivery components facilitate application of the active sunscreen component to the wearer's skin such that the active sunscreen component is distributed across a protected area of the wearer's skin in an effective coverage amount. The sunscreen assists in coral reef recovery, as well as improving other ocean environments, and thereby promotes ocean health.

### RELATED APPLICATION INFORMATION

This application claims priority to U.S. Provisional Application No. 63/052,304, entitled, “SUNSCREEN THAT PROMOTES OCEAN HEALTH,” filed on Jul. 15, 2020. The content of the above-noted application is incorporated by reference herein as if set forth in full and priority to this application is claimed to the full extent allowable under U.S. law and regulations.

### FIELD OF THE INVENTION

The present invention relates generally to environmentally friendly sunscreens and, in particular, to sunscreens that not only avoid damage to coral reefs and other ocean environments but actively promote ocean health.

### BACKGROUND OF THE INVENTION

In recent years, marine biologists have noted an alarming degradation of ocean environments, notably including the bleaching and dying of coral reefs. Depletion of coral reefs is of particular concern because, among other things, coral reefs play a significant role in sustaining biodiversity and protecting coastlines from damage due to waves and storms. Moreover, the destruction of coral reefs has a significant economic impact including lost income from food supplies and a threat to the eco-tourism industry. The preservation of coral reefs has therefore become a matter of intense international concern.

There are a number of factors that contribute to the depletion of coral reefs. Two factors that have drawn considerable attention are pollution, including residues from sunscreens, and ocean acidification associated with global warming. In particular, common ingredients in conventional sunscreens have been linked to coral bleaching and many ingredients in conventional sunscreens are detrimental to marine life. Some ingredients associated with coral bleaching have been banned in some jurisdictions. Ocean acidification is closely related to global warming and, therefore, has been harder for coastal authorities to address. The problem of coral depletion and other impacts on marine life, as well as efforts to address these concerns, are described in more detail in the Provisional Application.

### SUMMARY OF THE INVENTION

The present invention is directed to a sunscreen that promotes ocean health, especially for coral reefs. Research has shown that conventional sunscreens have had a devastating impact on coral reefs. In particular, certain conventional sunscreen ingredients have been shown to contribute to coral bleaching and, unfortunately, visitors to coral reefs have proved to be very effective in distributing sunscreen residues across coral environments. Some jurisdictions have now outlawed sunscreens including coral bleaching agents. While these laws are expected to slow the destruction of coral reefs, such reefs remain in need of assistance to begin recovery.

The present invention is directed to assisting in coral reef recovery, as well as improving other ocean environments, and thereby promotes ocean health. This is accomplished in a couple of ways. First, the invention takes advantage of the potential influence that visitors can have on coral reef environments by providing a sunscreen that includes ingredients to promote ocean health. These ingredients may include agents to maintain ocean alkalinity and thereby combat ocean acidification, especially in the vicinity of reefs. Reef visitors, who have long contributed to coral reef destruction, are thereby deployed as a delivery system to assist in reef recovery. The inventive sunscreen also includes minerals that increase ocean pH, which increases the buffer capacity of the ocean water, increasing the alkalinity and the total dissolved inorganic carbon, thereby increasing carbonate ions. Carbonate ions are taken from the ocean when carbon dioxide reacts with the ocean forming carbonic acid. Thus, increasing greenhouse gases have removed carbonate ions from the ocean but the carbonate ions that are being depleted. Thus, by contributing carbonate ions to the water, the sunscreen is increasing ocean pH directly around the swimmer (combatting ocean acidification) and adding carbonate ions for the corals to better reproduce. The invention also promotes ocean health by educating those likely to have an impact on coral reefs as to the threats to reefs, the resulting impact on the greater ocean environment, and how individuals can help. The invention also provides an effective sunscreen without using ingredients that are harmful to the wearer's health.

In accordance with one aspect of the present invention, a sunscreen is provided that promotes ocean health. The sunscreen includes an active sunscreen component, a delivery component, and one or more alkalinity enhancing components. The sunscreen product can increase the pH of ambient water, e.g., in the vicinity of a reef. Additional ingredients may be provided for color, scent, skin feel, to promote skin health, or other attributes. Though reef safe ingredients are preferred, alkalinity enhancing components can be added to conventional sunscreen formulations to provide some benefit in combating acidification of the ocean environment.

The active sunscreen component preferably includes a broadband sunscreen agent effective to filter or block UV-A and UV-B radiation. The active component can be provided in different formulations or concentrations to achieve different SPF values as may be desired. In preferred formulations, the active component includes a mineral sunscreen agent such as zinc oxide and or titanium dioxide. Although any sunscreen agents can be used in accordance with the present invention, including any conventional sunscreen agents, reef safe ingredients are preferred. In this regard, when zinc oxide or titanium dioxide are employed, they are preferably provided in non-nano form as nano forms have been shown to accumulate in and harm reefs.

The delivery component facilitates application of the active sunscreen component to the wearer's skin such that the active sunscreen component is distributed across a protected area of the wearer's skin in an effective coverage amount. In this regard, the sunscreen may be provided as a lotion, spray, gel, a stick (e.g., an anhydrous stick), a water in oil formula, an oil in water emulsion, or foam and the nature of the delivery component may vary depending on the nature of the sunscreen. In one formulation, the sunscreen is provided as a lotion and the delivery component includes a blend of beneficial oils and butters substantially free from ingredients that may penetrate into the wearer's bloodstream and have deleterious effects on health. For example, the delivery component may include one or more of sunflower oil, mango butter, cocoa butter, and beeswax.

The alkalinity enhancing components can improve the resistance of ambient water to acidification, increase water pH, and contribute carbonate ions to the water. Research has shown an increasing acidification, or lowering of the pH, of ocean waters in recent decades. This is believed to be due to the increased absorption of carbon from the atmosphere that forms carbonic acid in the ocean. Some studies have shown that ocean acidification can compromise the health of coral reefs. The alkalinity enhancing components enhance the ability of ocean water to resist such acidification, improve the reef's ability to calcify, and are otherwise benign or have a positive impact on the health of coral reefs. Any such ingredients, or combinations thereof, can be employed as alkalinity enhancing components in accordance with the present invention. Examples include certain mineral salts, chalks, or limes, such as magnesium carbonate and/or calcium carbonate. In addition, silicates or minerals containing magnesium or calcium may be included as an alkalinity enhancing component. For example, olivine may be added to the sunscreen as it contains magnesium and quickly breaks down to release the magnesium into the ocean environment. A further example of an alkalinity enhancing component is arginine, an amino acid that may affect water pH. In general, and ingredient that directly or indirectly affects pH can be utilized. The alkalinity enhancing components are preferably provided in an amount sufficient to have an impact, in the collective, on coral reefs and the ocean environment, while remaining within safe ranges for application to the wearer's skin.. Preferably, the alkalinity enhancing components are provided in a concentration of at least 5% by weight of the sunscreen product, for example, between about 5-30% and, more preferably, between about 10 to 20% by weight, for example, about 15%.

In one exemplary formulation, the active sunscreen component is zinc oxide. The zinc oxide concentration may be between about 20 to 30% by weight of the sunscreen product. The delivery component may constitute between about 25 to 50% by weight of the sunscreen product, for example, about 35.6%. In one formulation the delivery component includes sunflower oil (between about 15 to 30% by weight, for example, about 23%), mango butter (between about 4 to 10% by weight, for example, about 6.8%), cocoa butter (between about 2 to 5% by weight, for example, about 3.5%), and beeswax (between about 1 to 3%, for example, about 2.1%). The alkalinity enhancing component may include a combination of magnesium carbonate (between about 2 to 6%, for example, about 3.9%) and calcium carbonate (between about 8 to 20%, for example, about 11.5%). Further details of the sunscreen product, alternative formulations, and preparation techniques are provided below.

### BRIEF DESCRIPTION OF THE DRAWING

For a more complete understanding of the present invention and further advantages thereof, reference is now made to the following detailed description, taken in conjunction with the drawings, in which:

FIG. 1 is a flow chart illustrating a process for making sunscreen products in accordance with the present invention.

### DETAILED DESCRIPTION OF THE INVENTION

The present invention relates to sunscreens that promote ocean health. In the following description, the invention as set forth in the context of specific formulations for specific sunscreen products, e.g., sticks, creams, and lotions. However, it will be appreciated that a variety of sunscreen products can be produced in accordance with the present invention. Moreover, the specific formulations may be varied for a variety of reasons including: to provide different SPF ratings; to provide different appearances, fragrances, and skin feel; to comply with laws or regulations; and to optimize ocean health and user health in view of evolving conditions and understanding. Accordingly, the following description should be understood as exemplary and not by way of limitation.

The sunscreen products of the present invention may generally be understood as including four categories of ingredients; a sunscreen component; and alkalinity enhancing component; a delivery component; and additives. Each of these is described in more detail below. It will be appreciated that some ingredients may fall into more than one category, e.g., an ingredient used for a delivery component or as an additive to achieve a more desirable product may also provide some sunscreen or alkalinity enhancing property. Accordingly, the following categories are provided to facilitate description of exemplary products and not to imply limitations.

### 1. Sunscreen Components

A primary purpose of the sunscreen component is to filter or block some amount of harmful solar radiation from affecting the wearer's skin. This may involve absorbing and/or reflecting UV radiation. Preferably, the sunscreen component includes one or more ingredients that collectively function as a broadband sunscreen to filter or block at least UV-A and UV-B radiation. The sunscreen products may include products with different radiation blocking effectiveness or SPF ratings, for example, SPF ratings of SPF 30 and SPF 50.

Another important property of the sunscreen component of the present invention is to promote the health of ocean environments as well as the health of the wearer. In this regard, the sunscreen component is preferably reef-safe and preferably free from ingredients that pose health risks to wearers.

Preferred sunscreens in this regard include one or more mineral sunscreen agents such as zinc oxide and titanium oxide. The agents typically act as a physical sunscreen rather than a chemical sunscreen. The zinc oxide and/or titanium oxide are preferably provided in a non-nano form to avoid accumulation in reefs.

### 2. Alkalinity and/or pH Enhancing Components

A primary purpose of the alkalinity enhancing components is to counteract ocean acidification, or raise the pH, of the local environment, e.g., in the vicinity of reefs. In this regard, it is noted that sunscreen components do not stay on the wearer's skin but, rather, become dissolved or suspended in the surrounding environment, for example, ocean environments near reefs. Indeed, this accounts for the injury to reefs from sunscreens as has been observed by researchers. In this regard, it is not necessary for the sunscreen components to be dissolved or suspended in amounts sufficient to alter the chemistry of substantial ocean volumes. Rather, such components, even in small amounts, can affect sensitive micro-environments in the vicinity of reefs or other vulnerable ocean areas. In particular, high-traffic areas such as reefs can be affected by sunscreens.

Accordingly, the inventive sunscreens include an alkalinity enhancing component that improves the resistance of the nearby ambient water to acidification or raises the pH of nearby ambient water. Any ingredient that tends to raise the pH of the nearby ambient water can be used in this regard. Examples include certain mineral salts, chalks, amino acids or limes. In addition, silicates or minerals containing magnesium or calcium may be included as an alkalinity enhancing component. For example, olivine may be added to the sunscreen as it contains magnesium and quickly breaks down to release the magnesium. Other examples include various sources of carbonate ions, for example, magnesium carbonate and/or calcium carbonate. Preferably, the alkalinity enhancing components are provided in a concentration of at least 5% by weight of the sunscreen product and, more preferably, between about 10 to 30% by weight, for example, about 20%.

### 3. Delivery Components

A primary purpose of the delivery components is to facilitate application of the active sunscreen component to the wearer's skin such that the active sunscreen component is distributed across a protected area of the wearer's skin in an effective coverage amount. In addition, the delivery components may enable the release of the alkalinity enhancing components to the surrounding environment. Moreover, the delivery components may be selected to avoid ingredients that may penetrate into the wearer's bloodstream and have deleterious effects on health. The nature of the delivery components depends, in part, on the type of sunscreen product, for example, a lotion, spray, gel, foam, stick, or other product. In the specific formulations described below, the delivery components are selected to yield a cream, a lotion, and a stick.

### 4. Additives

The sunscreen product may also include a number of additives to provide a more desirable commercial product. For example, the sunscreen product may include ingredients for improved texture, skin feel, fragrance, color, or the like as well as stabilizing agents or other ingredients to improve shelf life or provide a more practical commercial product.

### 5. Examples

a. SPF 30 Anhydrous Stick

The following is an example of the sunscreen product in accordance with the present invention that may be provided as an anhydrous stick. Such a stick may be rubbed on the wearer's skin to apply the sunscreen product as a film that can be absorbed into the wearer's skin. Ranges are provided for each ingredient. The ranges provided are determined by weight and reflect a range of compositions that are believed to provide a suitable product in accordance with the present invention. In some cases, ranges of complementary items are, to some extent, dependent on one another. For example, the calcium carbonate and magnesium carbonate should collectively comprise at least 5% by weight of the product and may comprise, for example, about 20% of the product. Preferably, the products include between about 2-6% magnesium carbonate and between about 2-20% of calcium carbonate by weight. Moreover, the ranges below, in some cases, contemplate an SPF 30 product and the active sunscreen component may be varied for other SPF ratings.
Zinc Oxide; 5-30%, for example, about 20% for SPF 30 product
Cocos nucifera (Coconut) Oil; 10-30%
Caprilic/Capric Triglyceride; 10-25%
Polyhydroxystearic Acid; 5-20%
Theobroma cacoa (Cocoa) Butter; 5-10%
Helianthus annuus (Sunflower) Seed Wax; 5-15%
Calcium Carbonate; 1-10%
Magnesium Carbonate; 1-10%
Diisostearyl Polyglyceryl—3 Dimer Dilinoleate; 0.5-5%
Tocopherol; 0.1-1%
Bisabolol; 0.1-1%
Zingiber officinale (Ginger) Root Extract; 0.1-1%

The ingredients are combined in the desired proportions and heated, for example, to about 80-85 degrees Celsius while gently stirring or mixing. The mixture can then be poured into the product container and allowed to cool to form the desired stick consistency.

b. Water in Oil Lotion SPF 30

The following formulation can be used to provide a sunscreen product in the form of a lotion that can be applied to the wearer's skin. Again, ranges are provided by weight and, in some cases, the ranges for complementary components are interdependent.
Zinc Oxide; 5-30%, for example, about 20% for SPF 30
Water; 20-50%
Caprilic/Capric Triglyceride; 10-20%
Ethyl Palmate; 5-10%
Calcium Carbonate; 1-5%
Magnesium Carbonate; 1-5%
Pyrus malus (Apple) Fruit Extract; 1-5%
Glycerin; 1-5%
Magnesium Sulfate; 1-5%
Helianthus annuus (Floor) Seed Wax; 0.5-3%
Shorea Robusta Resin; 0.1-2%
Xanthan Gum; 0.1-1%
Sodium Benzoate; 0.1-1%
Citric Acid; 0.1-1%
Bisbolol; 0.1-1%
Zingiber officinale (Ginger) Root Extract; 0.1-1%
Tocopherol; 0.1-1%
Tocopherol; 0.1-1%
Polyglyceryl-4 Diisostearate/Polyhydroxystearate/Sebacate; 1-5%
Polyglyceryl-3 Oleate; 1-5%
Diisostearyl Polyglyceryl-3 Dimer Dilinoleate; 1-5%

The ingredients are combined and stirred or blended until well-mixed. The mixture may be heated to facilitate mixing but does not necessarily need to be heated as much as in the case of the Anhydrous stick. The mixture can then be poured into product containers, allowed to cool, and sealed/packaged for shipping.

c. Oil in Water Cream SPF 30

The following formulation can be used to produce an oil in water cream. Ranges are provided by weight and, as noted above, the ranges for certain ingredients may be interdependent.
Zinc Oxide; 5-30%, for example, about 20% for SPF 30
Water; 30-60%
Mangifera indica (Mango) Seed Butter; 1-10%
Ethyl Palmate; 1-10%
Caprilic/Capric Triglyceride; 1-10%
Calcium Carbonate; 1-5%
Magnesium Carbonate; 1-5%
Lactobacillus Ferment; 1-5%
Pyrus malus (Apple) Fruit Extract; 1-5%
Glycerin; 1-5%
Diisostearyl Polyglyceryl-3 Dimer Dilineolate; 0.5-2%
Magnesium Aluminum Silicate; 0.1-1%
Xanthan Gum; 0.1-1%
Tocolpherol; 0.1-1%
Bisabolol; 0.1-1%
Zingiber officinale (Ginger) Extract; 0.1-1%
Lactic Acid; 0.1-1%
Arachidyl Alcohol; 1-5%
Behenyl Alcohol; 1-5%
Arachidyl Glucoside; 1-5%

The ingredients are combined and stirred or blended until well mixed. Again, the mixture may be heated to promote mixing but does not necessarily need to be heated to the same extent as the anhydrous stick. The mixture is then poured into a product container, allowed to cool, sealed, and packaged for shipping.

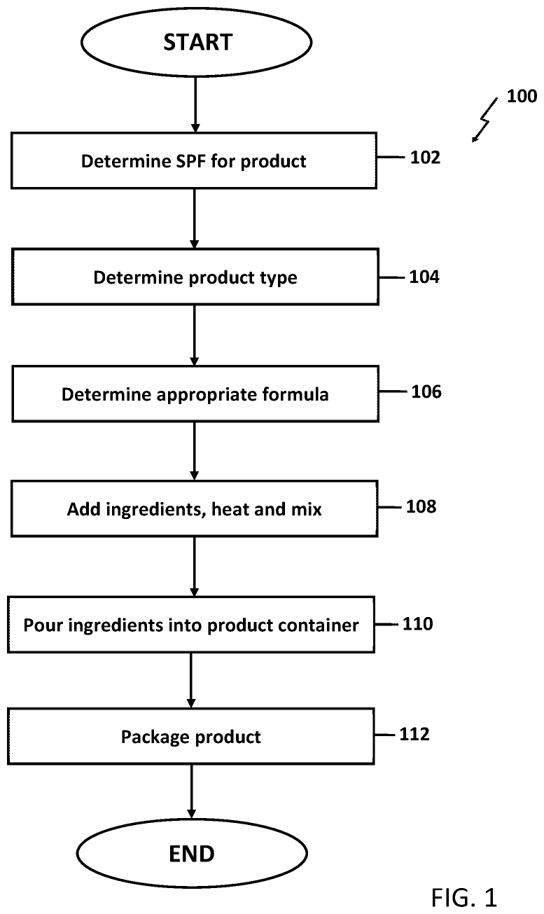

FIG. 1 is a flowchart illustrating a process 100 for producing a sunscreen product in accordance with the present invention. The illustrated process 100 is initiated by determining (102) an SPF for the product. For example, products may be provided with an SPF of SPF 30 or SPF 50. It will be appreciated that the SPF rating will be used in determining the amount of active sunscreen component that is added to the product.

The illustrated process 100 further involves determining (104) the product type. As noted above, in accordance with the present invention, sunscreens may be provided in various product types such as lotions, sprays, gels, foams, and sticks among others. As described above, different formulations can be provided for different product types. Moreover, the production process may differ for different product types. For example, to produce a stick-type product, the ingredients may require more heating to get the ingredients to blend and then may require more cooling for the product to set-up.

Based on the desired SPF rating and the product type, an appropriate formula may be determined (106) for the product. The ingredients may then be added (108) together in a mixing vessel such as a vat, heated, and stirred or blended until the ingredients are well-mixed. As noted above, the ingredients may include a reef-safe active sunscreen component, and alkalinity enhancing component, delivery components, and additives. The ingredients may then be poured (110) into a product container and cooled. The nature of the product container may vary depending on the type of product. For example, a stick product may be poured into an elongate stick housing. For a cream type product, the mixture may be poured into a squeeze tube. For lotions, the mixture may be poured into a bottle or spray bottle. Finally, the product may be packaged (112) for shipping. Preferably, the product container and packaging are formed from recycled materials to the extent possible. For example, recycled plastic materials harvested at least in part from ocean waste would further promote the objectives of the invention relating to enhancing ocean health.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed:

1. A sunscreen product, comprising:
an active sunscreen component for protecting a wearer's skin from ultraviolet radiation;
a delivery component for facilitating application of the active sunscreen component to the wearer's skin such that the active sunscreen component is distributed across a protected area of the wearer's skin in an effective coverage amount; and
one or more alkalinity enhancing components to improve the resistance of ambient water to acidification, said alkalinity components comprising at least 5% by weight of said sunscreen product.

2. The sunscreen product of claim 1, wherein said active sunscreen component comprises a mineral sunscreen agent.

3. The sunscreen product of claim 2, wherein said active sunscreen component comprises one or both of zinc oxide and titanium dioxide.

4. The sunscreen product of claim 3, wherein said active sunscreen component is provided in a concentration of between 5-30% by weight of said sunscreen product.

5. The sunscreen product of claim 1, wherein said delivery component renders said sunscreen product in the form of one of a spray, a lotion, a gel, and a foam.

6. The sunscreen product of claim 1, wherein said delivery component comprises between 25-50% by weight of said sunscreen product.

7. The sunscreen product of claim 1, wherein said alkalinity enhancing component comprises between 5 to 30% by weight of said sunscreen product.

8. The sunscreen product of claim 1, wherein said alkalinity enhancing component comprises at least one of magnesium carbonate and calcium carbonate.

9. The sunscreen product of claim 1, wherein said alkalinity enhancing component comprises between about 2 to 6% by weight magnesium carbonate and between about 2 to 20% by weight calcium carbonate.
